(19) United States
(12) Patent Application Publication
(10) Pub. No.: US 2026/0367199 A1
(43) Pub. Date: Nov. 19, 2026

(54) DIFFUSION-BASED ADAPTIVE E-SKIN GEOMAGNETIC DETECTION AND REFLEX GENERATION

(71) Applicant: INTERNATIONAL BUSINESS MACHINES CORPORATION, Armonk, NY (US)

(72) Inventors: Aaron Keith BAUGHMAN, Cary, NC (US); Amit Kumar RAHA, Barrackpore (IN); Kavitha Hassan YOGARAJ, Bangalore (IN); Christian EGGENBERGER, Wil (CH)

(21) Appl. No.: 19/208,758

(22) Filed: May 15, 2025

(51) Int. Cl. G01R 33/10; G01R 33/00; G01R 33/022; G01R 33/07

(52) U.S. Cl. CPC G01R 33/10; G01R 33/0047; G01R 33/022; G01R 33/072

(57) ABSTRACT

Computer implemented methods, systems, and computer program products include program code executing on a processor(s) utilizes sensors of an electronic skin device to detect magnetic fields within a local detection vicinity of the device. The processor(s) identifies, within the local detection vicinity, additional electronic skin devices based on obtaining electronic signatures (the devices identify other electronic skin devices) and these devices form a swarm. The processor(s) adds the electronic skin device to the swarm. The processor(s) communicates between the devices comprising the swarm. The processor(s) generates a computing object that includes distances between each of the electronic skin devices comprising the swarm, strength of magnetic fields of magnetic objects detected by the swarm devices, and velocity of the magnetic objects. The processor(s) triggers a reflexive reaction in at least one electronic skin device in the swarm when the device is within a pre-determined proximity of a magnetic object.

### BACKGROUND

One or more aspects relate, in general, to improving sensing ranges.

Electronic skin or e-skin is a thin, flexible material that can be attached to surfaces, including human skin, and is designed to mimic the way human skin senses and transmits information. E-skins include sensors that measure external stimuli like temperature and pressure and transmit electrical signals to indicate what has been sensed. E-skin is implemented as flexible circuits that provide interfaces which are not rigid. E-skins have established themselves as a versatile technology to restore or enhance human perception and potentially enable softer robotics.

The Hall effect is the production of a voltage difference across an electrical conductor that is transverse to an electric current in the conductor and to an applied magnetic field perpendicular to the current. Specifically, the Hall coefficient is defined as the ratio of the induced electric field to the product of the current density and the applied magnetic field. Hence, it is a characteristic of the material from which the conductor is made, since its value depends on the type, number, and properties of the charge carriers that constitute the current.

Diffusion-based mapping is a technique in data analysis that uses a model of data as a random walk to uncover the underlying structure of the data. It's a non-linear dimensionality reduction method that maps high-dimensional data to lower-dimensional spaces while preserving the non-linear relationships. This is achieved by leveraging the concept of what is referred to as a diffusion distance, which quantifies the relatedness of data points based on their connectivity, rather than just their distance.

Geometric detection refers to detection or measurement of the earth's magnetic field.

A swarm of devices refers to a group of interconnected devices working collectively to achieve a common goal. The word swarm is used because it is derived from collective behavior of animals such as bees or birds. The devices comprising a swarm can include, but are not limited to personal computing devices, e-skins, Internet of things devices, etc. Each device comprising a swarm can provide similar or different capabilities or functionalities when compared to others, but when working in a swarm, these devices coordinate their actions to perform tasks more efficiently than a single device.

### SUMMARY

Shortcomings of the prior art are overcome, and additional advantages are provided through the provision of a computer-implemented method for improving reflex reactions to stimuli for individuals in a given vicinity. The method includes utilizing, by one or more processors comprising an electronic skin device, sensors of the electronic skin device to detect magnetic fields within a local detection vicinity of the electronic skin device; based on detecting the magnetic fields, identifying, by the one or more processors, within the local detection vicinity, additional electronic skin devices based on obtaining electronic signatures from the additional electronic skin devices, wherein each of the additional electronic skin devices had also identified any other electronic skin devices, wherein the additional electronic skin devices and the other electronic skin devices form a swarm; adding, by the one or more processors, the electronic skin device to the swarm; communicating, by the one or more processors, between the electronic skin devices comprising the swarm; based on the communicating, generating, by the one or more processors, a computing object, wherein the computing object comprises the electronic skin devices comprising the swarm and distances between each of the electronic skin devices comprising the swarm, strength of magnetic fields of magnetic objects detected by each the electronic skin devices comprising the swarm, and velocity of the magnetic objects; and triggering, by the one or more processors, a reflexive reaction in at least one electronic skin device of the electronic skin devices comprising the swarm, based on determining that the at least one electronic skin device is within a pre-determined proximity of a magnetic object of the magnetic objects in the computing object.

Shortcomings of the prior art are overcome, and additional advantages are provided through the provision of a computer program product for improving reflex reactions to stimuli for individuals in a given vicinity. The computer program product comprises a storage medium readable by one or more processors and storing instructions for execution by the one or more processors for performing a method. The method includes, for instance: utilizing, by the one or more processors which comprise an electronic skin device, sensors of the electronic skin device to detect magnetic fields within a local detection vicinity of the electronic skin device; based on detecting the magnetic fields, identifying, by the one or more processors, within the local detection vicinity, additional electronic skin devices based on obtaining electronic signatures from the additional electronic skin devices, wherein each of the additional electronic skin devices had also identified any other electronic skin devices, wherein the additional electronic skin devices and the other electronic skin devices form a swarm; adding, by the one or more processors, the electronic skin device to the swarm; communicating, by the one or more processors, between the electronic skin devices comprising the swarm; based on the communicating, generating, by the one or more processors, a computing object, wherein the computing object comprises the electronic skin devices comprising the swarm and distances between each of the electronic skin devices comprising the swarm, strength of magnetic fields of magnetic objects detected by each the electronic skin devices comprising the swarm, and velocity of the magnetic objects; and triggering, by the one or more processors, a reflexive reaction in at least one electronic skin device of the electronic skin devices comprising the swarm, based on determining that the at least one electronic skin device is within a pre-determined proximity of a magnetic object of the magnetic objects in the computing object.

Shortcomings of the prior art are overcome, and additional advantages are provided through the provision of a system for improving reflex reactions to stimuli for individuals in a given vicinity. The system includes: a memory, one or more processors in communication with the memory, and program instructions executable by the one or more processors via the memory to perform a method. The method can include for instance: utilizing, by the one or more processors which comprise an electronic skin device, sensors of the electronic skin device to detect magnetic fields within a local detection vicinity of the electronic skin device; based on detecting the magnetic fields, identifying, by the one or more processors, within the local detection vicinity, additional electronic skin devices based on obtaining electronic signatures from the additional electronic skin devices, wherein each of the additional electronic skin devices had also identified any other electronic skin devices, wherein the additional electronic skin devices and the other electronic skin devices form a swarm; adding, by the one or more processors, the electronic skin device to the swarm; communicating, by the one or more processors, between the electronic skin devices comprising the swarm; based on the communicating, generating, by the one or more processors, a computing object, wherein the computing object comprises the electronic skin devices comprising the swarm and distances between each of the electronic skin devices comprising the swarm, strength of magnetic fields of magnetic objects detected by each the electronic skin devices comprising the swarm, and velocity of the magnetic objects; and triggering, by the one or more processors, a reflexive reaction in at least one electronic skin device of the electronic skin devices comprising the swarm, based on determining that the at least one electronic skin device is within a pre-determined proximity of a magnetic object of the magnetic objects in the computing object.

Computer systems and computer program products relating to one or more aspects are also described and may be claimed herein. Further, services relating to one or more aspects are also described and may be claimed herein.

Additional aspects of the present disclosure are directed to systems and computer program products configured to perform the methods described above. Additional features and advantages are realized through the techniques described herein. Other embodiments and aspects are described in detail herein and are considered a part of the claimed aspects.

### BRIEF DESCRIPTION OF THE DRAWINGS

One or more aspects are particularly pointed out and distinctly claimed as examples in the claims at the conclusion of the specification. The foregoing and objects, features, and advantages of one or more aspects are apparent from the following detailed description taken in conjunction with the accompanying drawings in which:

FIG. 1 depicts one example of a computing environment to perform, include and/or use one or more aspects of the present disclosure;

FIG. 2 depicts various aspects of an electronic skin (e-skin) device which is utilized in various examples in the present disclosure;

FIG. 3 is a workflow that illustrates various aspects of some examples in the present disclosure;

FIG. 4 is a workflow that illustrates various aspects of some examples in the present disclosure;

FIG. 5 is an example of a computing object which is generated by the program code in various aspects of some examples in the present disclosure;

FIG. 6 is a workflow that illustrates various aspects of some examples in the present disclosure; and

FIG. 7 is an example of a computing object which is generated by the program code in various aspects of some examples in the present disclosure.

### DETAILED DESCRIPTION

The computer-implemented methods, computer program products, and systems described herein can generate an undirected graph where the edges are the distances between the edges and the vertices of the graph are a vector where this vector can represent, for example, magnetic strength of an object and/or velocity of an object. As will be discussed in greater detail herein, in some of the examples herein a user wears an e-skin and sensors comprising the e-skin detect one or more magnetic fields. In some examples, program code executing on one or more processors triggers sensors comprising the e-skin to detect one or more magnetic fields. In some of the examples herein, the e-skin(s) comprises a processor to handle data collected by sensors comprising the e-skin. The processor of an e-skin is responsible for decoding and processing the sensor information, which can then be transmitted for further use. The e-skin system can also include other components like a flexible, stretchy skin-like covering, a power supply, and various sensors. Regarding these sensors, the program code (executing on the processor of the e-skin) can utilize these sensors in the e-skin to detect a geo-magnetic direction of the one or more magnetic fields. The program code can inform the user of the geo-magnetic direction. In some examples, however, the program code can utilize the sensors and the fields they sensed to locate a device and the program code can add the device detected to a swarm of devices (detected by the program code via the sensors in the e-skin). For example, the program code can add the device to the swarm when the program code determines that the setting of the device are such that participation in the swarm was selected (e.g., the user opted the device into the swarm) and that the device senses additional e-skin devices. The program code can optionally inform the user as devices are sensed, including when the devices sensed are sensing additional e-skin devices. Thus, the program code, via the sensors in the e-skin as well as optionally additional devices, can identify all local magnetic fields in the swarm. In these examples, the devices comprising the swarm can work collectively to identify all local magnetic fields. The program code can generate a graph of connected devices, based on identifying the fields. The graph generated by the program code can be an undirected graph with edges which are the distances between the edges and vertices which are vectors representing aspects including, but not limited to, magnetic strength of an object and/or a velocity of the object.

Participation in the swarm formed in the examples herein is voluntary (e.g., based on intentional device settings). The program code in the examples herein creates the swarm for the purpose of sharing data related to stimuli in an environment among devices. Each e-skin user (e.g., wearer) in the examples herein can opt out of participation in the swarm. Thus, the swarm of devices is not formed automatically in the sense that a user will opt his or her device into participation. However, when a user enables their e-skin device to participate in a swarm, the individual device can access and benefit from, functionally, environmental data obtained by the remainder of the devices in the swarm. The e-skin devices can continually (collectively) learn from experiencing environmental stimuli and thus, a given device which has not experienced a given stimulus, based on the experience with this stimulus of one or more other devices in the swarm, can be provided with data enabling the device to anticipate this stimulus (e.g., a haptic alert can be produced by the program code such that the user is aware of the stimuli and is warned to avoid it). Thus, each device in a swarm at a given geographic location can benefit from experiences at that geographic locations of the remaining devices. Because the data utilized to generate alerts (which are described in detail herein), allowing users to anticipate and/or avoid potential hazards, is based on experiences of multiple devices, data relating to an individual e-skin is not stored in a manner that differentiates one device from another. Rather, collective data forms training data utilized by the swarm (as a whole). Additionally, no personally identifiable information related to users of the e-skins forming the swarm is utilized even as training data nor is it stored. Location data of individual e-skins (as it related to stimuli) is combined into training data and hence, the source (the individual e-skin) providing each data point is not stored, and if temporarily captured to enhance training data, is purged systematically. In examples which store these data (which are not all examples) before being purged, data is securely stored and only the data obtained from and provided by devices opted in by users are utilized in the examples herein. Users are essentially anonymous in system, as the swarm data is regarding reaction data to a given stimulus (grouped together) and not particular to any user. Based on a device being part of a swarm, the program code in the examples herein does not direct any collective action. Rather, when a given e-skin is at a location where, based on data from devices in the swarm there exists an environmental stimulus, the program code can provide alerts, including but not limited to haptic warnings (via the e-skin) to enables the user of a e-skin, who may not be focused on the stimulus, to react individually in a manner that avoids any potential hazards presented by the presence of the stimulus. Thus, individual devices are informed by the collected and analyzed experiences of other devices.

As described in greater detail herein, the examples herein include the formation and utilization of a computing systems comprising (communicatively) connected e-skin devices. Program code comprising the system generated a self-organizing reflex gradient for each e-skin (e.g., e-skin wearer or user), such that the computing system comprises individuals who, based on data received by the devices they utilize (e.g., the e-skins) can react to local stimulation individually based on a particle swarm optimization technique built on top of an undirected graph of these connected devices. Reflex gradients refer to variations in the strength or intensity of a reflex response across different regions or levels of the body. The program code determines desired strengths of reflex responses to stimuli such that individuals utilizing devices in the swarm can react, in an anticipatory and proportionally accurate manner, to stimuli. The reflex gradient generated by the program code in these examples is self-organizing because the interactions between the devices and their surroundings automatically (e.g. spontaneously) generate the reflex gradient. The connected e-skin devices and the reflex gradient(s) are utilized, for example, to improve user navigation and interaction with the (physical) environment. The reflex gradient enables users utilizing an e-skin which is part of the network to experience enhanced reflexes and awareness, including potentially reacting more quickly to potential hazards or approaching objects. These enhanced senses and reflexes are enabled when devices in the network sense local magnetic and generate small voltage charges using the Hall effect, which extends human senses by providing an additional input channel. For example, a magnetic field generated by a vehicle could warn pedestrians with e-skin devices of its approach, enhancing their reflexes. Users utilizing devices in the network can adapt their behavior based on collective experiences. The examples herein merge e-skin devices, which are an emerging technology, with human biological capabilities to enhance orientation and reflexes of users of these devices. Enhanced orientation and reflexes enable quick reactions and awareness of surroundings, which can be crucial in various situations (e.g., to maintain the safety of the individual).

The examples herein are inextricably tied to computed and directed to a practical application. The examples are inextricably tied to computing at least because they utilize computing technologies for the purpose of mapping networks and features of computing devices. Hence, the examples utilize computing devices and address a practical application that is relevant to computing technology. Specifically, the examples herein can utilize sensors in e-skins to generate a collective network of e-skin wearers, each with their own self-organized reflex gradient, hence generating a system where individuals can respond to local stimulation in a coordinated manner. Thus, the program code in the examples herein can generate a distributed sensing and response system that is adaptable to various environmental changes, a practical application for a computing technology.

Certain of the examples herein are also inextricably tied to computing because machine learning is integrated into various examples herein. Over time, as users continue to wear an e-skin and interact with their environments while wearing the skin, and adjust their reflex gradients, the swarm (generated by the program code as described above as well as in greater detail herein) can as a whole can learn to respond more effectively to different types of local stimulation. The examples herein provide more than existing approaches in part because the adaptability of the swarm can imbue the systems herein with improved reactions and decision-making in response to changing conditions.

Certain of the examples herein are directed to various practical applications in which enhanced (and self-learning) sensing abilities are beneficial. For example, certain aspects of the examples here can be utilized in safety systems which detect approaching vehicles, navigation aids for visually impaired individuals, and/or collaborative robotics to enable robotic devices to react to stimuli, including but not limited to the presence of individuals.

Various computing devices can be utilized by users to orient the users. E-skin devices, which are incorporated into various aspects of the examples herein, remove rigid interfaces and provide magnetoreception endpoints. Because e-skins are affixed to the skin of a user, they are extremely sensitive to outside magnetic fields. A user of an e-skin can utilize the e-skin (or, rather, the sensors comprising the e-skin) to localize directionality and velocity with respect to the relative environment. However, program code (executing on one or more processors including microprocessors integrated into the e-skin) receiving data from the e-skin in traditional approaches will not be able to provide a user with data providing to the user with absolute directionality with respect to the earth's magnetic field. This limitation increases the need for rigid devices despite the advantages presented by the e-skin. But e-skins provide various advantages in environmental sensing, because, for example, individuals rely on reflex speed to anticipate movement of objects before they occur or are sensed by one or more of their five traditional senses (seeing, smelling, taste, touching, and hearing). The local magnetic fields sensed by an e-skin can generate small voltage charges (e.g., Hall effect) that can extend these senses. As perceived by an e-skin, a vehicle could be surrounded by a magnetic sphere (the magnetic sphere would emanate from the vehicle) and based on perceiving this sphere, program code obtaining data from sensors comprising an e-skin could warn pedestrians that the distance it is approaching. This warning could be delivered by the e-skin itself (e.g., haptic feedback). An e-skin could generate a voltage sensation that creates a human reflex exemplifying a conscious awareness of an approaching object. As will be explained herein, the example described herein provides a practical application by interlacing global directionality and reflex generation functions to enable removal of rigid devices for localization while increasing the range of sensing moving objects.

Certain of the examples herein provide substantial advantages when compared to existing approaches to environmental sensing based on interlacing global directionality and reflex generation functions. As described herein, e-skin devices can be utilized to enhance sensing by self-organizing and reflex gradient generation to aid in user (wearer) response time to environmental stimuli. The e-skin can behave as nodes and measure distances between e-skins in a vicinity (e.g., in a swarm). As described herein, program code executing on one or more processors, including internal to an e-skin, generate a system based upon a network of e-skin devices that are interconnected and communicate wirelessly to share information and coordinate actions, including but not limited to, responses to stimuli. In certain examples, herein, individuals who wear e-skin can become a swarm (of devices) and can collectively adapt their responses based on the interactions among them and the changing environment, generating more effective reflex gradients over time. The devices working together as a swarm enable swarm-based global electromagnetic localization based on local effects and swarm-based magnetic gradient sharing for extended reflex generation. Although the network of e-skins (e.g., the swarm) can act collectively, each individual e-skin can still work to sense local magnetic fields. These e-skins can generate small voltage charges using the Hall Effect. Another advantage of the examples herein is that the program code can perform confidence based local localization based on environmental changes in sampled data. Confidence-based approaches, in general, are regarding as (more) comprehensive systems for orientation and sensing moving objects. In certain of the examples herein, a confidence-based system involves a confidence-based approach to local localization: e.g., each e-skin can estimate its position within its local environment based on sensed stimuli and interactions with neighboring devices. Also, the examples herein can perform diffusion model based electromagnetic denoising. For example, the program code can generate diffusion model predictions of environmental electromagnetic noise based on a series of images generated by the devices in the swarm.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random-access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

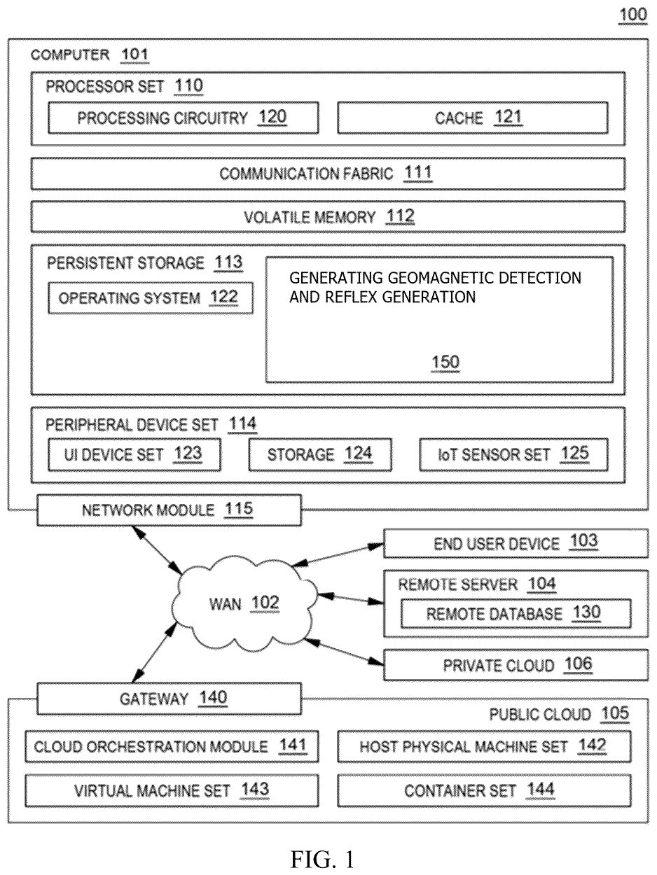

One example of a computing environment to perform, incorporate and/or use one or more aspects of the present disclosure is described with reference to FIG. 1. In one example, a computing environment 100 contains an example of an environment for the execution of at least some of the computer code involved in performing the inventive methods, such as a code block for generating geomagnetic detection and reflex generation 150. In addition to block 150, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 150, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

Computer 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

Processor set 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 150 in persistent storage 113.

Communication fabric 111 is the signal conduction path that allows the various components of computer 101 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

Volatile memory 112 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

Persistent storage 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid-state storage devices. Operating system 122 may take several forms, such as various known proprietary operating systems or open-source Portable Operating System Interface-type operating systems that employ a kernel. The code included in block 150 typically includes at least some of the computer code involved in performing the inventive methods.

Peripheral device set 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made though local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

Network module 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

End user device (EUD) 103 is any computer system that is used and controlled by an end user (for example, a customer of an enterprise that operates computer 101) and may take any of the forms discussed above in connection with computer 101. EUD 103 typically receives helpful and useful data from the operations of computer 101. For example, in a hypothetical case where computer 101 is designed to provide a recommendation and/or review to an end user, this recommendation would typically be communicated from network module 115 of computer 101 through WAN 102 to EUD 103. In this way, EUD 103 can display, or otherwise present, the recommendation and/or review to an end user. In some embodiments, EUD 103 may be a client device, such as thin client, heavy client, mainframe computer, desktop computer and so on.

Remote server 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation and/or review based on historical data, then this historical data may be provided to computer 101 from remote database 130 of remote server 104.

Public cloud 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

The examples herein include computer-implemented methods, computer program products, and computer systems where program code executing on one or more processors comprising an e-skin or communicatively coupled with an e-skin detect magnetic fields (of objects within a sensing vicinity of the e-skin). By sensing magnetic fields, e-skins identify potential stimuli in an environment. The program code can detect a geo-magnetic direction of these magnetic fields and/or determine the geo-magnetic direction of these magnetic fields. The program code adds the e-skin, which completed this detection, to a swarm of e-skin devices. The program code can inform a user of what is detected. The program code can identify all e-skin devices which comprise the swarm (and regularly update the device membership based on continuously monitoring the swarm). For example, each e-skin device comprising the storm can detect additional e-skin devices in their individual vicinities (e.g., based on magnetic fields) and the program code can continue to add newly discovered devices to the swarm. The program code can (continuously) identify each magnetic field in the swarm and utilize the identifies fields and their respective devices to generate and continuously update a graph of the interconnected devices (comprising the swarm). The program code, for example, can build an undirected graph with edges that are the distances between the devices and the vertices being a vector (of magnetic strengths of objects as velocities of the objects). Based on ethe vectors and the graphs generated by the program code, the program code can build a virtual map of all objects with a magnetic field in a vicinity of the swarm (e.g., including objects within the vicinity of the e-skin nodes and/or vertices comprising the swarm). The program code can utilize the virtual map to generate a set of solutions, including but not limited to a set of reflex gradients, in a solution space. The reflex gradient is based on the program code collecting (via the swarm) data related to objects and their magnetic fields and utilizing the magnetic fields of these objects to generate sets of directions from which the program code generates sets of the aforementioned reflex gradients. The program code can utilize Particle Swarm Optimization (PSO)/Accelerated Particle Swarm Optimization (APSO) to generate a self-organizing reflex gradient for each e-skin (e.g., for each individual wearing an e-skin). Thus, the individuals utilizing e-skin included in the swarm, based on the reflex gradients, can react simultaneously to local stimuli. The users can react based on the program code generating an alert to the e-skin which generates a reflexive reaction that is experienced by the user.

PSO refers to a computational algorithm inspired by the collective behavior of organisms like fish schooling or bird flocking, where decentralized agents interact locally to find an optimal solution for complex problems with discontinuous and non-linear parameters. In the examples herein, the decentralized agents are the e-skins (being worn by various individuals) and they work together to locate objects and inform members of the swarm about the objects in a manner that engenders a reflexive reaction to objects (e.g., via the Hall effect). PSO can be understood as an artificial intelligence (AI) technique and it can be used to find approximate solutions to extremely difficult or impossible numeric maximization and minimization problems (e.g., object location and object movement anticipation/prediction). PSO optimizes a problem by iteratively trying to improve a candidate solution regarding a given measure of quality. PSO is inspired by the collective behavior of social animals like birds flocking or fish schooling, where individuals cooperate to find optimal solutions. In PSO, each potential solution is represented as a particle, which moves through a multi-dimensional search space. Each particle has its own best-known position (personal best) and the swarm has a best-known position (global best). Particles update their positions and velocities based on both their personal best and the global best, guiding them towards better solutions. This iterative process continues until the swarm converges on a solution that meets the specified criteria.

The examples herein include a computer-implemented method for improving reflex reactions to stimuli for individuals in a given vicinity. In some of the examples herein, the program code, executing on the one or more processors, utilizes an electronic skin device, sensors of the electronic skin device to detect magnetic fields within a local detection vicinity of the electronic skin device. Based on detecting the magnetic fields, the program code identifies, within the local detection vicinity, additional electronic skin devices based on obtaining electronic signatures from the additional electronic skin devices, wherein each of the additional electronic skin devices had also identified any other electronic skin devices, wherein the additional electronic skin devices and the other electronic skin devices form a swarm. The program code adds the electronic skin device to the swarm. The program code communicates between the electronic skin devices comprising the swarm. Based on the communicating, the program code generates a computing object. The computing object comprises the electronic skin devices comprising the swarm and distances between each of the electronic skin devices comprising the swarm, strength of magnetic fields of magnetic objects detected by each of the electronic skin devices comprising the swarm, and velocity of the magnetic objects. The program code triggers a reflexive reaction in at least one electronic skin device of the electronic skin devices comprising the swarm, based on determining that the at least one electronic skin device is within a pre-determined proximity of a magnetic object of the magnetic objects in the computing object. This example is inextricably tied to computing, directed to a practical application, and provides more than existing approaches because it generates a collective network of e-skin wearers, each with their own self-organized reflex gradient, hence generating a system where individuals can respond to local stimulation in a coordinated manner. Thus, the program code generates a distributed sensing and response system that is adaptable to various environmental changes.

In some examples of the computer-implemented method, the computing object comprises a virtual map of magnetic fields. This example is inextricably tied to computing, directed to a practical application, and provides more than existing approaches because the virtual map enables an understandable perimeter for the sensing network, which is extended by the incorporation of the swarm.

In some examples of the computer-implemented method, the program code generating the computing object comprises the program code generating an undirected graph. The edges on the undirected graph comprise the distances, and wherein vertices of the undirected graph comprise vectors. Additionally, the program code can convert the undirected graph to a virtual map of magnetic fields. The computing object comprises the virtual map of magnetic fields. This example is inextricably tied to computing, directed to a practical application, and provides more than existing approaches at least because the undirected graph can be utilized to visualize to users the locations of various fields to test and improve the accuracy of the sensing of the swarm and hence, enable enhanced functionality for each e-skin.

In some examples of the computer-implemented method, the vectors are selected from the group consisting of: magnetic strength of the magnetic objects and velocity of the magnetic objects. This example is inextricably tied to computing, directed to a practical application, and provides more than existing approaches at least because the vectors in the undirected graph capture varied data to enable the similar enhanced sensing functionality for each e-skin within the swarm.

In some examples of the computer-implemented method, the converting comprises: the program code generating, based on the undirected graph, the virtual map. The virtual map is a virtual map of the magnetic objects. The magnetic objects are located in a locality of the electronic skin devices comprising the swarm. This example is inextricably tied to computing, directed to a practical application, and provides more than existing approaches at least because the program code generates a virtual map that enables visualization of the operational area of a swarm, which is generated in the examples herein. The virtual map also can be utilized (and continually improved) by additional e-skins which join the swarm generated in the examples herein.

In some examples of the computer-implemented method, the virtual map comprises a confidence percentage related to an object type for each magnetic object of the magnetic objects. This example is inextricably tied to computing, directed to a practical application, and provides more than existing approaches at least because through utilization of e-skins comprising the swarm, confidence levels can be adjusted in order to continually improve the accuracy of the system generated by the program code such that it operates more efficiently and effectively than existing geographical sensing approaches.

In some examples of the computer-implemented method, the program code generating the undirected graph comprises: the program code deploying a function, to identify the additional electronic skin devices. Coordinates representing a location of each additional electronic skin device of the additional electronic skin devices. The program code can also apply a cost function to the coordinates to determine costs of graphs comprising the returned coordinates based on distances of the returned coordinates from the electronic skin device. Additionally, the program code can score, based on the costs of the graphs, the graphs to identify a graph with a best scored layout. The program code can implement the best scored layout as the undirected graph. This example is inextricably tied to computing, directed to a practical application, and provides more than existing approaches at least because by locating e-skin devices the system generated by the program code herein can grow in efficacy and accuracy and thus, continue to expand and improve the already improved approach to sensing.

In some examples of the computer-implemented method, the program code generating the virtual map of the magnetic objects comprises: the program code generating a set of solutions in space represented by the virtual map, wherein the set of solutions comprise: reflex gradients and a set of directions. This example is inextricably tied to computing, directed to a practical application, and provides more than existing approaches at least because the program code generates a virtual map that enables visualization of the operational area of a swarm, which is generated in the examples herein and provide additionally a reflex gradient to enable triggering of reactive responses for wearers of e-skins, which can assist to accommodate any individual who may have an impairment.

In some examples of the computer-implemented method, the program code triggering is based on a reflex gradient of the reflex gradients and directions selected from the set of directions, wherein the reflex gradient and the directions relate to the magnetic object of the magnetic objects. This example is inextricably tied to computing, directed to a practical application, and provides more than existing approaches at least because the use of the reflex gradient can control the reactions, including their intensity, from individuals approaching objects, hence guarding the safety of these individuals beyond the capabilities of their own senses.

In some examples of the computer-implemented method, the program code triggering generates a reflexive reaction experienced by a wearer of the electronic skin device based on the reflex gradient and the directions, wherein the magnetic object is anticipated stimuli for the wearer. This example is inextricably tied to computing, directed to a practical application, and provides more than existing approaches at least because the reactivity of an individual to stimuli can be informed not only by the experiences of the individual but by the program code mapping and machine learning from activities within the entirety of the swarm. Thus, each individual who wears an e-skin which is part of the swarm can benefit from the utility of the whole.

In some examples of the computer-implemented method, the program code generating the reflex gradients comprises: the program code utilizing Particle Swarm Optimization or Accelerated Particle Swarm Optimization to generate a reflex gradient for each electronic skin device of the electronic skin devices comprising the swarm, where the reflex gradients are self-organizing. This example is inextricably tied to computing, directed to a practical application, and provides more than existing approaches at least because integrating PSO and PPSO enhances the reflex gradient to increase its functionality and the ability of each element in the swarm to react to environmental stimuli.

In some examples of the computer-implemented method, the program code triggering comprises alerting at least one wearer of the at least one electronic skin device of a perceived immediate issue, wherein the alert is delivered based on triggering a Hall effect reaction. This example is inextricably tied to computing, directed to a practical application, and provides more than existing approaches at least because the Hall effect is integrated into the systems herein in order to create a reflective reaction (machine learned trained to a degree based on iterative data) for each user utilizing e-skins within the swarm.

In some examples of the computer-implemented method, the program code identifying, within the local detection vicinity, additional electronic skin devices comprises the program code identifying only devices which have settings enabled for identification and where the other electronic skin devices have the settings enabled for identification. This example is inextricably tied to computing, directed to a practical application, and provides more than existing approaches at least because enabling users of e-skins to opt in and out of participating in the swarm orchestrated by the program code increasing user involvement in choosing the benefits discussed herein.

The examples herein include a computer system for improving reflex reactions to stimuli for individuals in a given vicinity. The computer system can include a memory. The computer system can include one or more processors in communication with the memory, wherein the computer system is configured to perform a method. In this method, the program code, executing on the one or more processors, utilizes an electronic skin device, sensors of the electronic skin device to detect magnetic fields within a local detection vicinity of the electronic skin device. Based on detecting the magnetic fields, the program code identifies, within the local detection vicinity, additional electronic skin devices based on obtaining electronic signatures from the additional electronic skin devices, wherein each of the additional electronic skin devices had also identified any other electronic skin devices, wherein the additional electronic skin devices and the other electronic skin devices form a swarm. The program code adds the electronic skin device to the swarm. The program code communicates between the electronic skin devices comprising the swarm. Based on the communicating, the program code generates a computing object. The computing object comprises the electronic skin devices comprising the swarm and distances between each of the electronic skin devices comprising the swarm, strength of magnetic fields of magnetic objects detected by each of the electronic skin devices comprising the swarm, and velocity of the magnetic objects. The program code triggers a reflexive reaction in at least one electronic skin device of the electronic skin devices comprising the swarm, based on determining that the at least one electronic skin device is within a pre-determined proximity of a magnetic object of the magnetic objects in the computing object. This example is inextricably tied to computing, directed to a practical application, and provides more than existing approaches because it generates a collective network of e-skin wearers, each with their own self-organized reflex gradient, hence generating a system where individuals can respond to local stimulation in a coordinated manner. Thus, the program code generates a distributed sensing and response system that is adaptable to various environmental changes.

In some examples of the system, the computing object comprises a virtual map of magnetic fields. This example is inextricably tied to computing, directed to a practical application, and provides more than existing approaches because the virtual map enables an understandable perimeter for the sensing network, which is extended by the incorporation of the swarm.

In some examples of the system, the program code generating the computing object comprises the program code generating an undirected graph. The edges on the undirected graph comprise the distances, and wherein vertices of the undirected graph comprise vectors. Additionally, the program code can convert the undirected graph to a virtual map of magnetic fields. The computing object comprises the virtual map of magnetic fields. This example is inextricably tied to computing, directed to a practical application, and provides more than existing approaches at least because the undirected graph can be utilized to visualize to users the locations of various fields to test and improve the accuracy of the sensing of the swarm and hence, enable enhanced functionality for each e-skin.

In some examples of the system, the vectors are selected from the group consisting of: magnetic strength of the magnetic objects and velocity of the magnetic objects. This example is inextricably tied to computing, directed to a practical application, and provides more than existing approaches at least because the vectors in the undirected graph capture varied data to enable the similar enhanced sensing functionality for each e-skin within the swarm.

In some examples of the system, the converting comprises: the program code generating, based on the undirected graph, the virtual map. The virtual map is a virtual map of the magnetic objects. The magnetic objects are located in a locality of the electronic skin devices comprising the swarm. This example is inextricably tied to computing, directed to a practical application, and provides more than existing approaches at least because the program code generates a virtual map that enables visualization of the operational area of a swarm, which is generated in the examples herein. The virtual map also can be utilized (and continually improved) by additional e-skins which join the swarm generated in the examples herein.

In some examples of the system, the virtual map comprises a confidence percentage related to an object type for each magnetic object of the magnetic objects. This example is inextricably tied to computing, directed to a practical application, and provides more than existing approaches at least because through utilization of e-skins comprising the swarm, confidence levels can be adjusted in order to continually improve the accuracy of the system generated by the program code such that it operates more efficiently and effectively than existing geographical sensing approaches.

In some examples of the system, the program code generating the undirected graph comprises: the program code deploying a function, to identify the additional electronic skin devices. Coordinates representing a location of each additional electronic skin device of the additional electronic skin devices. The program code can also apply a cost function to the coordinates to determine costs of graphs comprising the returned coordinates based on distances of the returned coordinates from the electronic skin device. Additionally, the program code can score, based on the costs of the graphs, the graphs to identify a graph with a best scored layout. The program code can implement the best scored layout as the undirected graph. This example is inextricably tied to computing, directed to a practical application, and provides more than existing approaches at least because by locating e-skin devices the system generated by the program code herein can grow in efficacy and accuracy and thus, continue to expand and improve the already improved approach to sensing.

In some examples of the system, the program code generating the virtual map of the magnetic objects comprises: the program code generating a set of solutions in space represented by the virtual map, wherein the set of solutions comprise: reflex gradients and a set of directions. This example is inextricably tied to computing, directed to a practical application, and provides more than existing approaches at least because the program code generates a virtual map that enables visualization of the operational area of a swarm, which is generated in the examples herein and provide additionally a reflex gradient to enable triggering of reactive responses for wearers of e-skins, which can assist to accommodate any individual who may have an impairment.

In some examples of the system, the program code triggering is based on a reflex gradient of the reflex gradients and directions selected from the set of directions, wherein the reflex gradient and the directions relate to the magnetic object of the magnetic objects. This example is inextricably tied to computing, directed to a practical application, and provides more than existing approaches at least because the use of the reflex gradient can control the reactions, including their intensity, from individuals approaching objects, hence guarding the safety of these individuals beyond the capabilities of their own senses.

In some examples of the system, the program code triggering generates a reflexive reaction experienced by a wearer of the electronic skin device based on the reflex gradient and the directions, wherein the magnetic object is anticipated stimuli for the wearer. This example is inextricably tied to computing, directed to a practical application, and provides more than existing approaches at least because the reactivity of an individual to stimuli can be informed not only by the experiences of the individual but by the program code mapping and machine learning from activities within the entirety of the swarm. Thus, each individual who wears an e-skin which is part of the swarm can benefit from the utility of the whole.

In some examples of the system, the program code generating the reflex gradients comprises: the program code utilizing Particle Swarm Optimization or Accelerated Particle Swarm Optimization to generate a reflex gradient for each electronic skin device of the electronic skin devices comprising the swarm, where the reflex gradients are self-organizing. This example is inextricably tied to computing, directed to a practical application, and provides more than existing approaches at least because integrating PSO and PPSO enhances the reflex gradient to increase its functionality and the ability of each element in the swarm to react to environmental stimuli.

In some examples of the system, the program code triggering comprises alerting at least one wearer of the at least one electronic skin device of a perceived immediate issue, wherein the alert is delivered based on triggering a Hall effect reaction. This example is inextricably tied to computing, directed to a practical application, and provides more than existing approaches at least because the Hall effect is integrated into the systems herein in order to create a reflective reaction (machine learned trained to a degree based on iterative data) for each user utilizing e-skins within the swarm.

In some examples of the system, the program code identifying, within the local detection vicinity, additional electronic skin devices comprises the program code identifying only devices which have settings enabled for identification and where the other electronic skin devices have the settings enabled for identification. This example is inextricably tied to computing, directed to a practical application, and provides more than existing approaches at least because enabling users of e-skins to opt in and out of participating in the swarm orchestrated by the program code increasing user involvement in choosing the benefits discussed herein.

The examples herein include a computer program product for improving reflex reactions to stimuli for individuals in a given vicinity. The computer program product can include one or more computer readable storage media and program instructions collectively stored on the one or more computer readable storage media readable by at least one processing circuit. When executing the program instructions, the program code performs a method. In this program code, executing on the processing circuit, utilizes an electronic skin device, sensors of the electronic skin device to detect magnetic fields within a local detection vicinity of the electronic skin device. Based on detecting the magnetic fields, the program code identifies, within the local detection vicinity, additional electronic skin devices based on obtaining electronic signatures from the additional electronic skin devices, wherein each of the additional electronic skin devices had also identified any other electronic skin devices, wherein the additional electronic skin devices and the other electronic skin devices form a swarm. The program code adds the electronic skin device to the swarm. The program code communicates between the electronic skin devices comprising the swarm. Based on the communicating, the program code generates a computing object. The computing object comprises the electronic skin devices comprising the swarm and distances between each of the electronic skin devices comprising the swarm, strength of magnetic fields of magnetic objects detected by each of the electronic skin devices comprising the swarm, and velocity of the magnetic objects. The program code triggers a reflexive reaction in at least one electronic skin device of the electronic skin devices comprising the swarm, based on determining that the at least one electronic skin device is within a pre-determined proximity of a magnetic object of the magnetic objects in the computing object. This example is inextricably tied to computing, directed to a practical application, and provides more than existing approaches because it generates a collective network of e-skin wearers, each with their own self-organized reflex gradient, hence generating a system where individuals can respond to local stimulation in a coordinated manner. Thus, the program code generates a distributed sensing and response system that is adaptable to various environmental changes.

In some examples of the computer program product, the computing object comprises a virtual map of magnetic fields. This example is inextricably tied to computing, directed to a practical application, and provides more than existing approaches because the virtual map enables an understandable perimeter for the sensing network, which is extended by the incorporation of the swarm.

In some examples of the computer program product, the program code generating the computing object comprises the program code generating an undirected graph. The edges on the undirected graph comprise the distances, and wherein vertices of the undirected graph comprise vectors. Additionally, the program code can convert the undirected graph to a virtual map of magnetic fields. The computing object comprises the virtual map of magnetic fields. This example is inextricably tied to computing, directed to a practical application, and provides more than existing approaches at least because the undirected graph can be utilized to visualize to users the locations of various fields to test and improve the accuracy of the sensing of the swarm and hence, enable enhanced functionality for each e-skin.

In some examples of the computer program product, the vectors are selected from the group consisting of: magnetic strength of the magnetic objects and velocity of the magnetic objects. This example is inextricably tied to computing, directed to a practical application, and provides more than existing approaches at least because the vectors in the undirected graph capture varied data to enable the similar enhanced sensing functionality for each e-skin within the swarm.

In some examples of the computer program product, the converting comprises: the program code generating, based on the undirected graph, the virtual map. The virtual map is a virtual map of the magnetic objects. The magnetic objects are located in a locality of the electronic skin devices comprising the swarm. This example is inextricably tied to computing, directed to a practical application, and provides more than existing approaches at least because the program code generates a virtual map that enables visualization of the operational area of a swarm, which is generated in the examples herein. The virtual map also can be utilized (and continually improved) by additional e-skins which join the swarm generated in the examples herein.

In some examples of the computer program product, the virtual map comprises a confidence percentage related to an object type for each magnetic object of the magnetic objects. This example is inextricably tied to computing, directed to a practical application, and provides more than existing approaches at least because through utilization of e-skins comprising the swarm, confidence levels can be adjusted in order to continually improve the accuracy of the system generated by the program code such that it operates more efficiently and effectively than existing geographical sensing approaches.

In some examples of the computer program product, the program code generating the undirected graph comprises: the program code deploying a function, to identify the additional electronic skin devices. Coordinates representing a location of each additional electronic skin device of the additional electronic skin devices. The program code can also apply a cost function to the coordinates to determine costs of graphs comprising the returned coordinates based on distances of the returned coordinates from the electronic skin device. Additionally, the program code can score, based on the costs of the graphs, the graphs to identify a graph with a best scored layout. The program code can implement the best scored layout as the undirected graph. This example is inextricably tied to computing, directed to a practical application, and provides more than existing approaches at least because by locating e-skin devices the system generated by the program code herein can grow in efficacy and accuracy and thus, continue to expand and improve the already improved approach to sensing.

In some examples of the computer program product, the program code generating the virtual map of the magnetic objects comprises: the program code generating a set of solutions in space represented by the virtual map, wherein the set of solutions comprise: reflex gradients and a set of directions. This example is inextricably tied to computing, directed to a practical application, and provides more than existing approaches at least because the program code generates a virtual map that enables visualization of the operational area of a swarm, which is generated in the examples herein and provide additionally a reflex gradient to enable triggering of reactive responses for wearers of e-skins, which can assist to accommodate any individual who may have an impairment.

In some examples of the computer program product, the program code triggering is based on a reflex gradient of the reflex gradients and directions selected from the set of directions, wherein the reflex gradient and the directions relate to the magnetic object of the magnetic objects. This example is inextricably tied to computing, directed to a practical application, and provides more than existing approaches at least because the use of the reflex gradient can control the reactions, including their intensity, from individuals approaching objects, hence guarding the safety of these individuals beyond the capabilities of their own senses.

In some examples of the computer program product, the program code triggering generates a reflexive reaction experienced by a wearer of the electronic skin device based on the reflex gradient and the directions, wherein the magnetic object is anticipated stimuli for the wearer. This example is inextricably tied to computing, directed to a practical application, and provides more than existing approaches at least because the reactivity of an individual to stimuli can be informed not only by the experiences of the individual but by the program code mapping and machine learning from activities within the entirety of the swarm. Thus, each individual who wears an e-skin which is part of the swarm can benefit from the utility of the whole.

In some examples of the computer program product, the program code generating the reflex gradients comprises: the program code utilizing Particle Swarm Optimization or Accelerated Particle Swarm Optimization to generate a reflex gradient for each electronic skin device of the electronic skin devices comprising the swarm, where the reflex gradients are self-organizing. This example is inextricably tied to computing, directed to a practical application, and provides more than existing approaches at least because integrating PSO and PPSO enhances the reflex gradient to increase its functionality and the ability of each element in the swarm to react to environmental stimuli.

In some examples of the computer program product, the program code triggering comprises alerting at least one wearer of the at least one electronic skin device of a perceived immediate issue, wherein the alert is delivered based on triggering a Hall effect reaction. This example is inextricably tied to computing, directed to a practical application, and provides more than existing approaches at least because the Hall effect is integrated into the systems herein in order to create a reflective reaction (machine learned trained to a degree based on iterative data) for each user utilizing e-skins within the swarm.

In some examples of the computer program product, the program code identifying, within the local detection vicinity, additional electronic skin devices comprises the program code identifying only devices which have settings enabled for identification and where the other electronic skin devices have the settings enabled for identification. This example is inextricably tied to computing, directed to a practical application, and provides more than existing approaches at least because enabling users of e-skins to opt in and out of participating in the swarm orchestrated by the program code increasing user involvement in choosing the benefits discussed herein.

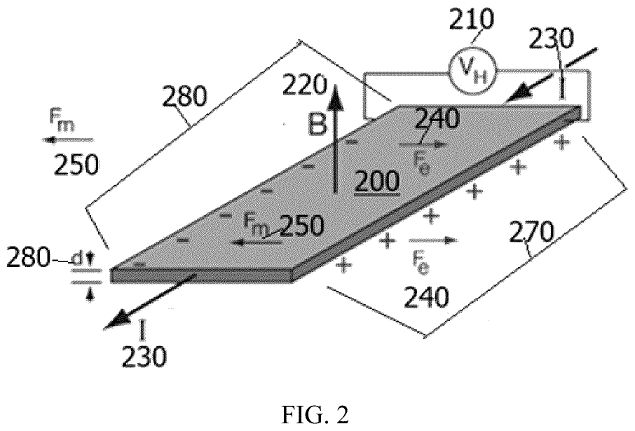

FIG. 2 is a workflow 200 that illustrates various aspects of an e-skin; as aforementioned, e-skins form systems (networks, swarms) to provide certain of the functionality described in some of the examples herein. The e-skin 200 (or rather, the portion displayed) is illustrated as if rigid although an e-skin 200 itself is flexible. Hence, the e-skin 200 is shown in a somewhat rigid configuration for illustrative purposes only. The e-skin is a Hall effect device; Hall effect sensors are used in various applications, such as detecting the position of a magnet, measuring current, and determining the presence of magnetic fields. In FIG. 2, V_{H} 210 represents Hall voltage (VH). The Hall effect is a phenomenon where a voltage difference, known as the Hall voltage (VH), develops across a conductor when a magnetic field is applied perpendicular to the direction of current flow. This voltage is proportional to the magnetic field strength and the drift velocity of the charge carriers in the conductor. Hence, VH is the potential difference produced across an electrical conductor when an external magnetic field is applied perpendicular to the current through the conductor. The way that VH works is that when current flows through a conductor in a magnetic field, the magnetic field exerts a force on the moving charge carriers (e.g., electrons). This force pushes the electrons to one side of the conductor, creating a charge separation. The Hall voltage is therefore the potential difference between the sides of the conductor due to the charge separation. VH is calculated with the formula VH=Bvd, B (e.g., B 220) is the magnetic field strength, v is the drift velocity of the charges, and d is the thickness of the conductor. Common applications of the Hall effect are measuring magnetic fields (which is performed in the examples herein) as VH is proportional to the magnetic field, enabling measurement of the strength of the magnetic field. VH can also be used to measure carrier density in materials with one type of charge carrier (e.g., metals). Hall effect devices, such as the e-skin 200 of FIG. 200 can be used to detect a position of a magnet, to measure current, and/or to determine the presence of magnetic fields (as in the present examples).

FIG. 2 illustrates the Hall voltage, V_{H} 210, a magnetic field B 220, and also illustrates I 230, the direction of a conventional current. Conventional current is defined as the direction in which positive charges would move if they were free to do so. In a circuit, conventional current flows from the positive terminal of the power source to the negative terminal (opposing the actual flow of electrons, which move from negative to positive). Fₑ 240 is the electric force from charge buildup, while Fₘ 250 is the magnetic force on negative charge carriers. The positive and negative sides of the e-skin 200 are also illustrated, with the negative charges 260 on the left and the positive charges 270 on the right. The thickness or density 280 is also illustrated in FIG. 2.

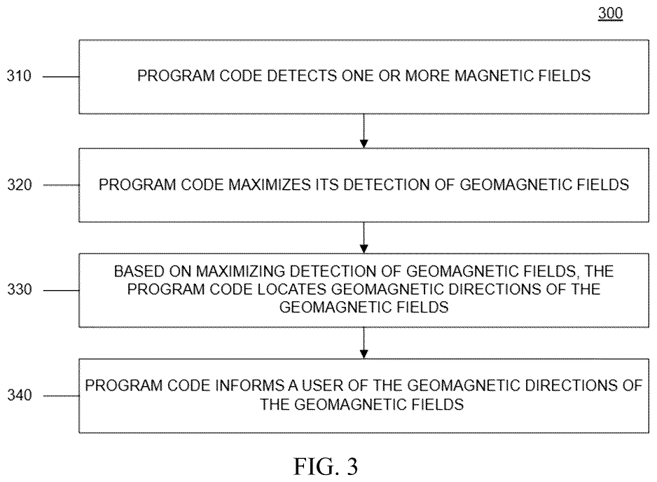

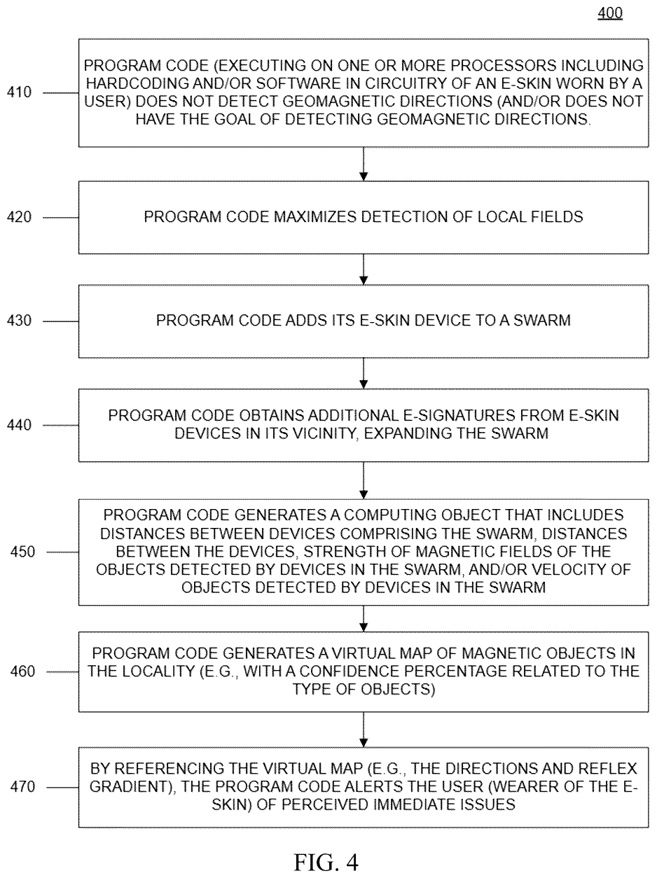

FIGS. 3 and 4 are workflows which illustrate various high-level aspects of some examples herein. These can be combined into various examples herein but are provided separately for clarity and illustrative purposes only. In both these examples, an e-skin device performs various activities to detect either geomagnetic fields or local magnetic fields. A geomagnetic field refers to the overall magnetic field of the Earth, generated by circulating currents in the molten iron core while local magnetic fields are the localized, smaller-scale magnetic fields that exist within the Earth's surface and can be influenced by geological formations, magnetic minerals, and external sources like currents in the ionosphere. A geomagnetic field resembles a bar magnet with magnetic poles located in the northern and southern hemispheres and it is not static and can change over time (e.g., secular variation), on scales varying from milliseconds to millions of years. A local magnetic field can vary in shape and can be complex and irregular, depending on the local geological and magnetic features. Changes in a local field can vary significantly from the geomagnetic field.

Referring to FIG. 3, in this workflow 300, the program code (executing on one or more processors including hardcoding and/or software in circuitry of an e-skin worn by a user) detects geomagnetic directions. A geomagnetic direction is the direction of the Earth's magnetic field at a specific location. The geomagnetic direction is not always aligned with true north (geographic north), and the angle between the geomagnetic direction and true (geographic) north is referred to as a magnetic declination. Magnetic declination is the difference between a direction in which a compass needle points and the direction to true north. Thus, the program code detects one or more magnetic fields (310). The program code maximizes its detection of geomagnetic fields (320). Based on maximizing detection of geomagnetic fields, the program code locates geomagnetic directions of the geomagnetic fields (330). The program code informs a user of the geomagnetic directions of the geomagnetic fields (340).

In contrast with the workflow 300 of FIG. 3, in the workflow 400 of FIG. 4, the program code (executing on one or more processors including hardcoding and/or software in circuitry of an e-skin worn by a user) does not detect geomagnetic directions (and/or does not have the goal of detecting geomagnetic directions. Thus, in FIG. 4, once the program code detects one or more magnetic fields (410), the program code maximizes detection of local fields (420). If and only if the e-skin has been configured by a user to opt into a swarm, the program code adds its e-skin device to the swarm (430) and as part of this swarm (which can be ever expanding) obtains additional e-signatures from e-skin devices in its vicinity, expanding the swarm (440). As aforementioned, a user can opt out of their e-skin participating in a swarm. However, provided that the e-skin was configured to opt into the swarm, once the program code has obtained all e-signatures within its vicinity, the initial e-skin device can operate in concert with the swarm. Based on operating in concert with the swarm, the program code identifies all local magnetic fields (identified by any device within the swarm as they are interconnected and sharing data). The program code generates a computing object that includes distances between devices comprising the swarm, distances between the devices, strength of magnetic fields of the objects detected by devices in the swarm, and/or velocity of objects detected by devices in the swarm (450). In some examples, the computing object is a graph. For example, the computing object can be an undirected graph with edges being the distance between the edges and vertices being a vector (e.g., magnetic strength of object, velocity of object).

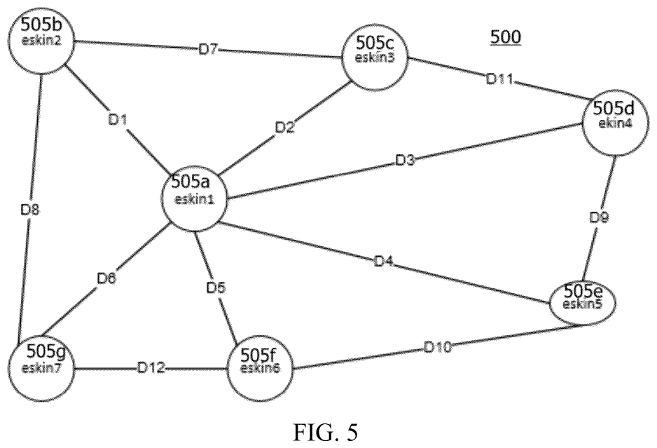

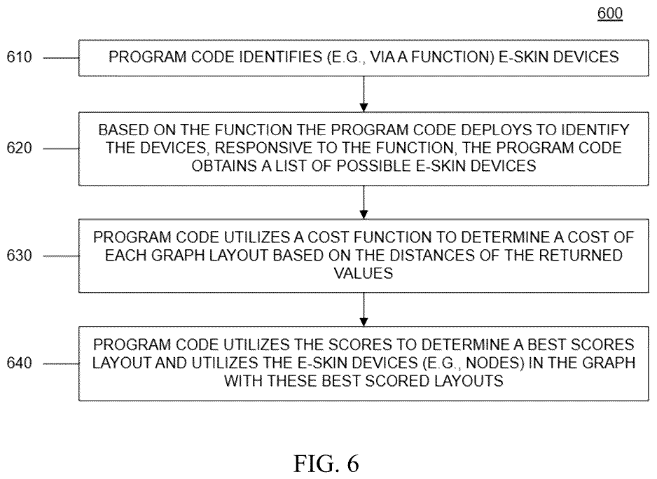

FIG. 5 is an example of a graph 500 generated by the program code. The graph comprises various e-skins 505a-505g and the distances D1-D11 between them. The program code generates this type of graph to build a swarm of e-skins with closely connected e-skins as a constraint. FIG. 6 is a workflow 600 that provides additional details in examples where the program code generates a graph (e.g., FIGS. 5, 500). The program code identifies (e.g., via a function) e-skin devices (610). Based on the function the program code deploys to identify the devices, responsive to the function, the program code obtains a list of possible e-skin devices (620). The program code mapped layout can be represented by the pseudocode below:
Map_layouts=generate_eskin_swarm.map_layouts(my_current_status)

Below is a list of coordinates in this example (provided for illustrative purposes only and not to introduce any limitations), which can be returned responsive to the program code utilizing a function to identify e-akin devices. These coordinates represent possible devices.
[[3, 2, 6, 7, 4, 5],
[7, 4, 6, 1, 2, 3],
[1, 4, 6, 7, 16, 12],
[12, 16, 6, 7, 4, 1],
[3, 5, 9, 8, 11, 14],
[14, 11, 9, 8, 5, 3],
[7, 16, 15, 12, 13, 14],
[7, 16, 13, 12, 15, 18],
[14, 13, 15, 12, 16, 7],
[14, 13, 16, 12, 15, 18],
[18, 15, 13, 12, 16, 7],
[18, 15, 16, 12, 13, 14],
[8, 11, 16, 14, 13, 12],
[8, 11, 13, 14, 16, 19],
[12, 13, 16, 14, 11, 8],
[12, 13, 11, 14, 16, 19],
[19, 16, 13, 14, 11, 8],
[19, 16, 11, 14, 13, 12],
[12, 15, 17, 18, 21, 23],
[23, 21, 17, 18, 15, 12],
[14, 16, 26, 19, 22, 25],
[25, 22, 26, 19, 16, 14],
[19, 22, 26, 25, 24, 23],
[23, 24, 26, 25, 22, 19]]

As aforementioned, a constraint of a swarm is distance. Thus, the program code utilizes a cost function to determine a cost of each graph layout based on the distances of the returned values (630). Hence, the cost function is utilized by the program code to select devices using the cost of each layout, which can be by default, a total error rate and distance to a first e-skin device observed, to find candidate e-skin that can be utilized in the swarm as a connected component. The cost function calculates a score based on distance. The program code can select a candidate score of connected e-skin devices to select a device. The pseudocode below represents how the program code can calculate a scores.
scores=generate_eskin_swarm.map_evaluate(my_current_status)

Below is a representation of devices located (based on mapped coordinates) couples with scores calculated by the program code. These examples are provided for illustrative purposes only.
(([3, 2, 6, 7, 4, 5], 0.1409544035570952),
([3, 2, 0, 1, 4, 7], 0.159886911767115),
([14, 11, 9, 8, 5, 3], 0.16797160130080224),
([19, 16, 13, 14, 11, 8], 0.1825119371865237),
([8, 11, 13, 14, 16, 19], 0.18331648549982482),
([7, 4, 0, 1, 2, 3], 0.19124485963881122),
([23, 24, 26, 25, 22, 19], 0.19226855309761348),
([25, 22, 20, 19, 16, 14], 0.19228399510047922),
([19, 22, 26, 25, 24, 23], 0.2000625493093675),
([14, 16, 20, 19, 22, 25], 0.20604403000055715),
([8, 11, 16, 14, 13, 12], 0.2580131332633393),
([12, 13, 16, 14, 11, 8], 0.27134706745983517),
([19, 16, 11, 14, 13, 12], 0.2755049869801992),
([12, 13, 11, 14, 16, 19], 0.2879346238104463),
([14, 13, 15, 12, 10, 7], 0.3474625243348848),
([1, 4, 6, 7, 10, 12], 0.34887580284018227),
([18, 15, 10, 12, 13, 14], 0.35020374737523874),
([7, 10, 15, 12, 13, 14], 0.35023196005467194),
([12, 10, 6, 7, 4, 1], 0.3628988750928549),
([18, 15, 13, 12, 10, 7], 0.39637978849009425),
([14, 13, 10, 12, 15, 18], 0.4063300698900274),
([23, 21, 17, 18, 15, 12], 0.41564717799937645),
([12, 15, 17, 18, 21, 23], 0.43370673744503807),
([7, 10, 13, 12, 15, 18], 0.4472384837396254)]

The program code utilizes the scores to determine a best scores layout and utilizes the e-skin devices (e.g., nodes) in the graph with these best scored layouts (640). For example, the program code can import a minimal cost flow (e.g., from ortools.graph.python import min_cost_flow). The program code can also determine a minimum cost flow (e.g., smcf=min_cost_flow. SimpleMinCostFlow( )). The program code generates an undirected graph (e.g., FIG. 5) using one or more acyclic graph algorithms. An acyclic graph is a graph that does not include any cycles or loops. In a directed acyclic graph (DAG), such as that depicted in FIG. 5, edges have a specific direction, and one cannot follow a path from one node back to itself, which is a property which allows for a natural ordering of the nodes in the graph, making it useful for applications like task scheduling and representing dependencies. The nodes in the graph of FIG. 5 are the e-skin devices 505a-505g. Similarly, a DAG algorithm is a graph algorithm where the edges have a direction and there are no cycles (loops) in the graph (as aforementioned relative to the DAG itself). Hence, one cannot follow edges and return to the starting node without revisiting other nodes. DAG algorithms are commonly used to model dependencies, precedence relationships, and the flow of information or tasks.

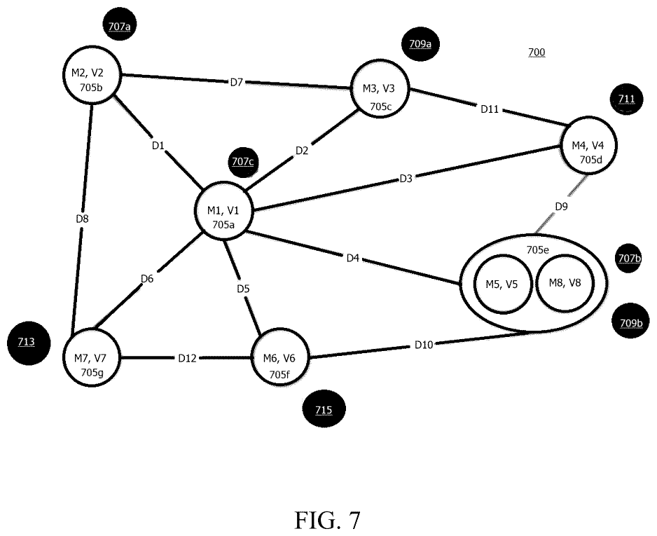

Returning to FIG. 4, the program code generates a virtual map of magnetic objects in the locality (e.g., with a confidence percentage related to the type of objects) (460). To generate the virtual map, the program code utilizes the object (e.g., directed graph), output an earlier portion of the workflow 400 as input into this portion. FIG. 7 is an example of a virtual map of magnetic objects in a locality 700. In FIG. 7, the e-skins 705a-705g are situated similarly to FIG. 5 to demonstrate how the results of the earlier workflow 400 aspect were utilized in this aspect. The virtual map generated by the program code includes the initial graph elements as well as objects with magnetic fields, which in this example include objects of a first type 707a-707c, objects of a second type 709a-709b, an object of a third type 711, an object of the fourth type 713, and an object of a fifth type 715. The magnetic fields M1, V1-M8, V8 of the objects are represented in their adjacent vertices. The object types can include but are not limited to electric cars (or other vehicles with magnetic fields), cellular phones or smart phones, mobile personal computing devices (e.g., laptops, portable gaming consoles), microwave ovens or other smart kitchen devices, and/or network access points (transmitters and/or receivers). The magnetic fields of the various objects are represented in the vertices of the virtual map of magnetic objects in a locality 700. The magnetic field of a first object 707a is represented as “M2, V2” in the second e-skin 705b. In some cases, the magnetic field of more than one object can be represented in a vertex, as a second object 707b, and a third object 709b, are represented as a first magnetic field M5, M5 in the vertex of the fifth e-skin 705e, and a second magnetic field M8, V8 in the vertex of the fifth e-skin 705e. As illustrated in FIG. 7, a swarm of e-skin devices can have multiple magnetics objects. Different objects can have fields of different strengths and hence can be detected by the e-skins comprising a swarm at different distances, thus, the program code of the e-skins can identify objects based on their field strengths.

As part of generating a virtual map of magnetic objects in the locality (e.g., with a confidence percentage related to the type of objects) (460), program code can generate a set of solutions into the space (e.g., the vicinity of the swarm). The program code obtains objects and their magnetic fields and utilizes the magnetic fields of objects to generate a set of directions. The program code can then generate sets of possible reflex gradients. The solution space generated by the program code can include reflex gradients (e.g., a set of reflex gradients), and a directions set comprising directions to transmit to users (individuals wearing the e-skins in the swarm), such that the e-skins can trigger reflexive reactions in the wearers to anticipated stimuli.

Also, as part of generating a virtual map of magnetic objects in the locality (e.g., with a confidence percentage related to the type of objects) (460), the program code can utilize PSO and/or APSO to generate reflex gradients, which are build self-organizing reflex gradients for each e-skin wearer. Taken together, the individual gradients form a system of individuals that react to local stimulation individually. Thus, the solution space, reflex gradients and direct sets, interlace both global directionality and reflex generation functions to enable removal of rigid devices for localization while increasing the range of sensing moving objects. As noted in FIG. 4, by referencing the virtual map (e.g., the directions and reflex gradient), the program code alerts the user (wearer of the e-skin) of perceived immediate issues (470). As each e-skin includes Hall effect sensors, the program code can trigger a Hall effect reaction based on stimuli.

In some examples, in order to improve the functionality of the swarm, the program code can utilize diffusion denoising. In these examples, the program code applies a diffusion model which takes an electromagnetic image over several time stamps and predicts electromagnetic noise. The program code can remove this predicted noise from the image and translate it back into recognizable electromagnetics. The diffusion denoising can be accomplished based on utilizing a trained neural network (NN). For example, the program code can develop and train machine learning models, including with underlying recurrent neural networks (RNNs) and/or convolutional neural network (CNNs), to generate denoise environmental data. The program code continuously updates and refines the model to improve accuracy and efficiency. As will be discussed herein, the program code generates, refines, and continuously tunes machine learning algorithms which are utilized by the program code to generate (and re-generate) images.

An NN is a biologically inspired programming paradigm which enables a computer to learn from data from disparate data sources. This learning is referred to as deep learning, which is a set of techniques for learning in neural networks. Neural networks, including modular neural networks, are capable of pattern recognition with speed, accuracy, and efficiency, in situations where data sets are multiple and expansive, including across a distributed network of the technical environment. Modern neural networks are non-linear statistical data modeling tools. They are usually used to model complex relationships between inputs and outputs or to identify patterns in data (i.e., neural networks are non-linear statistical data modeling or decision-making tools). In general, program code utilizing neural networks can model complex relationships between inputs and outputs and identify patterns in data. Because of the speed and efficiency of neural networks, especially when parsing multiple complex data sets, neural networks and deep learning provide solutions to many problems in image recognition, speech recognition, and natural language processing. It is the image recognition abilities which are particularly useful in the examples herein. Neural networks can model complex relationships between inputs and outputs to identify patterns in data, including in images, for classification. For this reason, machine learning models in the examples herein can utilize neural networks to generate a technical infrastructure design for a computing environment which the program code can, in some examples, implement and enforce.

In certain embodiments of the present invention the program code utilizes a CNN. CNNs are so named because they utilize convolutional layers that apply a convolution operation (a mathematical operation on two functions to produce a third function that expresses how the shape of one is modified by the other) to the input, passing the result to the next layer. The convolution emulates the response of an individual neuron to visual stimuli. Each convolutional neuron processes data only for its receptive field. It is generally not practical to utilize general (i.e., fully connected feedforward) neural networks to process data rich objects, as very high number of neurons would be necessary, due to the very large input sizes associated with larger files. Utilizing a CNN addresses this issue as it reduces the number of free parameters, allowing the network to be deeper with fewer parameters, as regardless of the file size, the CNN can utilize a consistent number of learnable parameters because CNNs fine-tune large amounts of parameters and massive pre-labeled datasets to support a learning process. CNNs resolve the vanishing or exploding gradients problem in training traditional multi-layer neural networks, with many layers, by using backpropagation. Thus, CNNs can be utilized in large-scale recognition systems, giving state-of-the-art results in segmentation, object detection, and object retrieval. Semantic recognition is an example of large-scale recognition in which a CNN can be utilized by the program code.

In certain embodiments of the present invention the program code utilizes an RNN. An RNN is a class of NN where connections between units form a directed cycle to exhibit dynamic temporal behavior. Unlike feedforward NNs, RNNs can use their internal memory to process arbitrary sequences of inputs. For this reason, current applications of RNNs include unsegmented data recognition, connected handwriting recognition, and speech recognition.

Various aspects and embodiments are described herein. Further, many variations are possible without departing from a spirit of aspects of the present disclosure. It should be noted that, unless otherwise inconsistent, each aspect or feature described and/or claimed herein, and variants thereof, may be combinable with any other aspect or feature.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description but is not intended to be exhaustive or limited to in the form disclosed. Many modifications and variations will be apparent to those of ordinary skill in the art. The embodiment was chosen and described to best explain various aspects and the practical application, and to enable others of ordinary skill in the art to understand various embodiments with various modifications as are suited to the particular use contemplated.

What is claimed is:

1. A computer-implemented method for improving reflex reactions to stimuli for individuals in a given vicinity, comprising:
utilizing, by one or more processors comprising an electronic skin device, sensors of the electronic skin device to detect magnetic fields within a local detection vicinity of the electronic skin device;
based on detecting the magnetic fields, identifying, by the one or more processors, within the local detection vicinity, additional electronic skin devices based on obtaining electronic signatures from the additional electronic skin devices, wherein each of the additional electronic skin devices had also identified any other electronic skin devices, wherein the additional electronic skin devices and the other electronic skin devices form a swarm;
adding, by the one or more processors, the electronic skin device to the swarm;
communicating, by the one or more processors, between the electronic skin devices comprising the swarm;
based on the communicating, generating, by the one or more processors, a computing object, wherein the computing object comprises the electronic skin devices comprising the swarm and distances between each of the electronic skin devices comprising the swarm, strength of magnetic fields of magnetic objects detected by each the electronic skin devices comprising the swarm, and velocity of the magnetic objects; and
triggering, by the one or more processors, a reflexive reaction in at least one electronic skin device of the electronic skin devices comprising the swarm, based on determining that the at least one electronic skin device is within a pre-determined proximity of a magnetic object of the magnetic objects in the computing object.

2. The computer-implemented method of claim 1, wherein the computing object comprises a virtual map of magnetic fields.

3. The computer-implemented method of claim 1, wherein generating the computing object comprises:
generating, by the one or more processors, an undirected graph, wherein edges on the undirected graph comprise the distances, and wherein vertices of the undirected graph comprise vectors; and
converting, by the one or more processors, the undirected graph to a virtual map of magnetic fields, wherein the computing object comprises the virtual map of magnetic fields.

4. The computer-implemented method of claim 3, wherein the vectors are selected from the group consisting of: magnetic strength of the magnetic objects and velocity of the magnetic objects.

5. The computer-implemented method of claim 3, wherein the converting comprises:
generating, by the one or more processors, based on the undirected graph, the virtual map, wherein the virtual map is a virtual map of the magnetic objects, wherein the magnetic objects are located in a locality of the electronic skin devices comprising the swarm.

6. The computer-implemented method of claim 5, wherein the virtual map comprises a confidence percentage related to an object type for each magnetic object of the magnetic objects.

7. The computer-implemented method of claim 3, wherein generating the undirected graph comprises:
deploying, by the one or more processors, a function, to identify the additional electronic skin devices, wherein coordinates representing a location of each additional electronic skin device of the additional electronic skin devices;
applying, by the one or more processors, a cost function to the coordinates to determine costs of graphs comprising the returned coordinates based on distances of the returned coordinates from the electronic skin device;
scoring, by the one or more processors, based on the costs of the graphs, the graphs to identify a graph with a best scored layout; and
implementing, by the one or more processors, the best scored layout as the undirected graph.

8. The computer-implemented method of claim 5, wherein generating the virtual map of the magnetic objects comprises:
generating, by the one or more processors, a set of solutions in space represented by the virtual map, wherein the set of solutions comprise: reflex gradients and a set of directions.

9. The computer-implemented method of claim 8, wherein the triggering is based on a reflex gradient of the reflex gradients and directions selected from the set of directions, wherein the reflex gradient and the directions relate to the magnetic object of the magnetic objects.

10. The computer-implemented method of claim 9, wherein the triggering generates a reflexive reaction experienced by a wearer of the electronic skin device based on the reflex gradient and the directions, wherein the magnetic object is anticipated stimuli for the wearer.

11. The computer-implemented method of claim 8, wherein generating the reflex gradients comprises:
utilizing, by the one or more processors, Particle Swarm Optimization or Accelerated Particle Swarm Optimization to generate a reflex gradient for each electronic skin device of the electronic skin devices comprising the swarm, wherein the reflex gradients are self-organizing.

12. The computer-implemented method of claim 1, wherein the triggering comprises alerting at least one wearer of the at least one electronic skin device of a perceived immediate issue, wherein the alert is delivered based on triggering a Hall effect reaction.

13. The computer-implemented method of claim 1, the identifying, within the local detection vicinity, additional electronic skin devices comprises identifying only devices which have settings enabled for identification and wherein the other electronic skin devices have the settings enabled for identification.

14. A computer system for improving reflex reactions to stimuli for individuals in a given vicinity, comprising:
a memory; and
one or more processors in communication with the memory, wherein the computer system is configured to perform a method, said method comprising
utilizing, by the one or more processors, wherein the one or more processors comprise an electronic skin device, sensors of the electronic skin device to detect magnetic fields within a local detection vicinity of the electronic skin device;
based on detecting the magnetic fields, identifying, by the one or more processors, within the local detection vicinity, additional electronic skin devices based on obtaining electronic signatures from the additional electronic skin devices, wherein each of the additional electronic skin devices had also identified any other electronic skin devices, wherein the additional electronic skin devices and the other electronic skin devices form a swarm;
adding, by the one or more processors, the electronic skin device to the swam;
communicating, by the one or more processors, between the electronic skin devices comprising the swarm;
based on the communicating, generating, by the one or more processors, a computing object, wherein the computing object comprises the electronic skin devices comprising the swarm and distances between each of the electronic skin devices comprising the swarm, strength of magnetic fields of magnetic objects detected by each the electronic skin devices comprising the swarm, and velocity of the magnetic objects; and
triggering, by the one or more processors, a reflexive reaction in at least one electronic skin device of the electronic skin devices comprising the swarm, based on determining that the at least one electronic skin device is within a pre-determined proximity of a magnetic object of the magnetic objects in the computing object.

15. The computer system of claim 14, wherein the computing object comprises a virtual map of magnetic fields.

16. The computer system of claim 14, wherein generating the computing object comprises:
generating, by the one or more processors, an undirected graph, wherein edges on the undirected graph comprise the distances, and wherein vertices of the undirected graph comprise vectors; and
converting, by the one or more processors, the undirected graph to a virtual map of magnetic fields, wherein the computing object comprises the virtual map of magnetic fields.

17. The computer system of claim 16, wherein the vectors are selected from the group consisting of: magnetic strength of the magnetic objects and velocity of the magnetic objects.

18. The computer system of claim 16, wherein the converting comprises:
generating, by the one or more processors, based on the undirected graph, the virtual map, wherein the virtual map is a virtual map of the magnetic objects, wherein the magnetic objects are located in a locality of the electronic skin devices comprising the swarm.

19. The computer system of claim 18, wherein the virtual map comprises a confidence percentage related to an object type for each magnetic object of the magnetic objects.

20. A computer program product for improving reflex reactions to stimuli for individuals in a given vicinity, the computer program product comprising:
one or more computer readable storage media and program instructions collectively stored on the one or more computer readable storage media readable by at least one processing circuit to:
utilize sensors of an electronic skin device to detect magnetic fields within a local detection vicinity of the electronic skin device;
based on detecting the magnetic fields, identify, within the local detection vicinity, additional electronic skin devices based on obtaining electronic signatures from the additional electronic skin devices, wherein each of the additional electronic skin devices had also identified any other electronic skin devices, wherein the additional electronic skin devices and the other electronic skin devices form a swarm;
add the electronic skin device to the swarm;
communicate between the electronic skin devices comprising the swarm;
based on the communicating, generate a computing object, wherein the computing object comprises the electronic skin devices comprising the swarm and distances between each of the electronic skin devices comprising the swarm, strength of magnetic fields of magnetic objects detected by each the electronic skin devices comprising the swarm, and velocity of the magnetic objects; and
trigger a reflexive reaction in at least one electronic skin device of the electronic skin devices comprising the swarm, based on determining that the at least one electronic skin device is within a pre-determined proximity of a magnetic object of the magnetic objects in the computing object.
